SEQUENCE LISTING (12 sequences)

SEQ ID NO: 1 - DNA, 27 bp, synthetic construct
  source 1..27: mol_type = other DNA
gtgagtgatggttgaggtagtgtggag

SEQ ID NO: 2 - DNA, 27 bp, synthetic construct
  source 1..27: mol_type = other DNA
  variation 12..19: note = d cannot be a if an adjacent d is a, if a d two positions away is a, or if a d three positions away is a; note = d is selected so that any two versions of SEQ ID NO: 2 differ in at least four positions; note = d cannot be g if any four adjacent d are each g
gtgagtgatggddddddddgtgtggag

SEQ ID NO: 3 - DNA, 35 bp, synthetic construct
  source 1..35: mol_type = other DNA
  variation 12..19: note = d cannot be g if any four adjacent d are each g; note = d cannot be a if an adjacent d is a, if a d two positions away is a, or if a d three positions away is a; note = d is selected so that any two versions of SEQ ID NO: 3 differ in at least four positions
gtgagtgatggddddddddgtgtggagnnnnnttt

SEQ ID NO: 4 - DNA, 35 bp, synthetic construct
  source 1..35: mol_type = other DNA
  variation 12..19: note = d is selected so that any two versions of SEQ ID NO:4 differ in at least four positions; note = d cannot be g if any four adjacent d are each g; note = d cannot be a if an adjacent d is a, if a d two positions away is a, or if a d three positions away is a
gtgagtgatggddddddddgtgtggagnnnnnggg

SEQ ID NO: 5 - DNA, 27 bp, synthetic construct
  source 1..27: mol_type = other DNA
  variation 18..25: note = d is selected so that any two versions of SEQ ID NO: 5 differ in at least four positions; note = d cannot be g if any four adjacent d are each g; note = d cannot be a if an adjacent d is a, if a d two positions away is a, or if a d three positions away is a
gtgagtgatggttgaggddddddddag

SEQ ID NO: 6 - DNA, 35 bp, synthetic construct
  source 1..35: mol_type = other DNA
  variation 18..25: note = d is selected so that any two versions of SEQ ID NO: 6 differ in at least four positions; note = d cannot be g if any four adjacent d are each g; note = d cannot be a if an adjacent d is a, if a d two positions away is a, or if a d three positions away is a
gtgagtgatggttgaggddddddddagnnnnnttt

SEQ ID NO: 7 - DNA, 35 bp, synthetic construct
  source 1..35: mol_type = other DNA
  variation 18..25: note = d cannot be g if any four adjacent d are each g; note = d is selected so that any two versions of SEQ ID NO: 7 differ in at least four positions; note = d cannot be a if an adjacent d is a, if a d two positions away is a, or if a d three positions away is a
gtgagtgatggttgaggddddddddagnnnnnggg

SEQ ID NO: 8 - DNA, 27 bp, synthetic construct
  source 1..27: mol_type = other DNA
gtgagtgatggttgaggttgtagttag

SEQ ID NO: 9 - DNA, 17 bp, synthetic construct
  source 1..17: mol_type = other DNA
gtgagtgatggttgagg

SEQ ID NO: 10 - DNA, 27 bp, synthetic construct
  source 1..27: mol_type = other DNA
  variation 14..24: map = d cannot be g if any four adjacent d are each g; note = d is selected so that any two versions of SEQ ID NO: 10 differ in at least four positions; note = d cannot be a if an adjacent d is a, if a d two positions away is a, or if a d three positions away is a
gtgagtgatggttdddddddddddgag

SEQ ID NO: 11 - DNA, 35 bp, synthetic construct
  source 1..35: mol_type = other DNA
  variation 14..24: note = d is selected so that any two versions of SEQ ID NO: 11 differ in at least four positions; note = d cannot be g if any four adjacent d are each g; note = d cannot be a if an adjacent d is a, if a d two positions away is a, or if a d three positions away is a
gtgagtgatggttdddddddddddgagnnnnnttt

SEQ ID NO: 12 - DNA, 35 bp, synthetic construct
  source 1..35: mol_type = other DNA
  variation 14..24: note = d cannot be a if an adjacent d is a, if a d two positions away is a, or if a d three positions away is a; note = d cannot be g if any four adjacent d are each g; note = d is selected so that any two versions of SEQ ID NO: 12 differ in at least four positions
gtgagtgatggttdddddddddddgagnnnnnggg

(19) United States
(12) Patent Application Publication
(10) Pub. No.: US 2026/0132455 A1
(43) Pub. Date: May 14, 2026

(54) METHODS AND COMPOSITIONS FOR AMPLIFYING AND IDENTIFYING GENOMIC DNA FROM SINGLE CELLS

(71) Applicants: The Curators of the University of Missouri, Columbia, MO (US); Board of Regents, the University of Texas System, Austin, TX (US)

(72) Inventor: Sen Xu, COLUMBIA, MO (US)

(21) Appl. No.: 19/382,524

(22) Filed: Nov. 7, 2025

Related U.S. Application Data
(60) Provisional application No. 63/718,271, filed on Nov. 8, 2024.

(51) Int. Cl. C12Q 1/6869; C12Q 1/6806; C12Q 1/686; C12Q 1/6876

(52) U.S. Cl. CPC C12Q 1/6869; C12Q 1/6806; C12Q 1/686; C12Q 1/6876; C12Q 2600/16

(57) ABSTRACT

A single-cell, whole genome sequence method is described. In an exemplary method, the method includes a first amplification step where a primer with a unique barcode is used to introduce the unique barcode into amplicons from a single cell. The amplicons can be further amplified in a second amplification stage, pooled into a single sequence library, sequenced and the sequencing results analyzed to identify nucleic acid sequences from individual single cells based on the unique barcode. The amplification stages of the method can include Multiple Annealing and Looping-Based Amplification Cycles (MALBAC). Also described herein are related primer collections and kits.

### CROSS-REFERENCE TO RELATED APPLICATION

This application claims the benefit of U.S. Provisional Patent Application Ser. No. 63/718,271, filed Nov. 8, 2024, the disclosure of which is incorporated herein by reference in its entirety.

### GOVERNMENT SUPPORT

This invention was made with government support under 2348390 awarded by the National Science Foundation. The government has certain rights in the invention.

### REFERENCE TO SEQUENCE LISTING SUBMITTED ELECTRONICALLY

The content of the electronically submitted sequence listing in XML format (Name: 3289_0013.xml; Size: 19,969 bytes; and Date of Creation: Nov. 4, 2025) filed with the application is incorporated herein by reference in its entirety.

### TECHNICAL FIELD

The presently disclosed subject matter relates to single cell, whole genome sequencing methods, as well as to primers and kits for performing the methods. In some embodiments, the method includes an amplification step comprising multiple annealing and looping-based amplification cycles (MALBAC).

### BACKGROUND

High-throughput single cell sequencing techniques have made enormous progress in the last couple of decades. Up to hundreds of thousands of cells can be assayed for genome-wide transcript abundance, chromatin accessibility, and epigenetic modification in one round of sequencing run. However, limited progress has been made for single-cell whole-genome sequencing (scWGS) and this progress is most applicable to select particular throughput levels. The most notable high-throughput scWGS techniques include a three-level combinatorial indexing method with potential throughput up to 1 million cells (Yin et al., 2019) and a microfluidic droplet barcoding method single with capacity of sequencing >50,000 cells (Lan et al., 2017). However, these high-throughput methods often require a special device (e.g., custom microfluidic devices) or a complicated workflow, which hampers the widespread adoption of these methods. At the low end of throughput for scWGS, each single cell undergoes whole genome amplification (WGA) with various WGA methods, sequencing library preparation, and genome sequencing. Although this kind of workflow is manageable and affordable when the throughput demand is low (e.g., dozens of cells), the cost of such an approach quickly becomes impractical when the throughput demand goes beyond hundreds of cells.

Accordingly, there is an ongoing need for additional single cell whole-genome sequencing methods, particularly methods for affordable, intermediate-level throughput single cell whole-genome sequencing.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a single-cell whole-genome sequencing method, the method comprising: (a) amplifying nucleic acid from each of a plurality of single cells, wherein the amplification is performed using one or more first stage primers that introduce a unique barcode region into amplicons from each of the plurality of single cells, wherein the unique barcode region introduced into the amplicons from each of the plurality of single cells differs from the unique barcode region introduced into amplicons from any other single cell in the plurality of single cells by at least three base pairs, thus providing amplicons from each of the plurality of single cells that are distinguishable based on the unique barcode assigned to each; (b) further amplifying the amplicons from each single cell using a second stage primer comprising the same unique barcode region as the one or more first stage primers used during amplification of nucleic acid from the same cell, thereby providing further amplified amplicons from each of the plurality of cells; (c) pooling the further amplified amplicons from each of the plurality of cells into a single sequencing library preparation; (d) sequencing the single sequencing library preparation to provide a library of sequencing reads; and (e) analyzing the sequencing reads to identify one or more nucleic acid sequences from one or more of the plurality of single cells based on the unique barcode sequence.

In some embodiments, the barcode region comprises: i) A, T, and G nucleotides but not C nucleotides; ii) at least three base pairs separating any two neighboring A nucleotides; and iii) no sequential G nucleotides of five or more in a row. In some embodiments, the unique barcode region introduced into the amplicons from each of the plurality of single cells differs from the unique barcode region introduced into amplicons from any other single cell in the plurality of single cells by at least four base pairs or by at least five base pairs.

In some embodiments, the unique barcode region is selected from the group comprising an 8mer, a 9mer, a 10mer, an 11mer, a 12mer, a 13mer, a 14mer, a 15mer, a 16mer, a 17mer, an 18mer, a 19mer and a 20mer. In some embodiments, the unique barcode region is an 11mer.

In some embodiments, for each individual single cell, the one or more first stage primers used in (a) and the second stage primer used in (b) each comprise the same common sequence, wherein the common sequence is a 27mer that comprises the unique barcode region. In some embodiments, the unique barcode is located anywhere in the 27mer common sequence from the fourth nucleotide from the 5′ end to the fourth nucleotide from the 3′ end. In some embodiments, the unique barcode is located in the 27-mer common sequence beginning at the fourteenth nucleotide from the 5′ end.

In some embodiments, the second amplification stage comprises polymerase chain reaction (PCR). In some embodiments, the method comprises Multiple Annealing and Looping-Based Amplification Cycles (MALBAC), wherein (a) comprises a pre-amplification stage comprising five cycles of linear amplification of an oligonucleotide of a single cell and (b) comprises a second amplification stage comprising PCR amplification.

In some embodiments, the presently disclosed subject matter provides an indexed MALBAC whole genome amplification (WGA) method for single-cell whole-genome sequencing, the method comprising: providing a plurality of single cells for which whole-genome sequencing is desired; providing a plurality of primer sets, wherein each primer set comprises one or more primers, wherein the one or more primers of each primer set comprise a unique barcoded region that differs from the unique barcoded region of primers in any other one of the plurality of primer sets by 3 or more nucleotides, and wherein a single primer set is assigned to each of the plurality of single cells; performing MALBAC WGA to amplify one or more oligonucleotides of each of the plurality of single cells, using a single primer set for the MALBAC WGA of each of the plurality of cells to thereby introduce a unique barcode region into each amplicon of the amplified oligonucleotides of each of the single cells; and pooling the barcoded amplicons from each of the single cells into a single sequencing library preparation, sequencing the library preparation to provide a plurality of sequencing reads, and analyzing the sequencing reads to identify genomic DNA sequences from single cells based on the unique barcode region. In some embodiments, the plurality of single cells comprises at least about 50 or more single cells. In some embodiments, the plurality of single cells comprises about 100 to about 1000 single cells.

In some embodiments, the presently disclosed subject matter provides a collection of primers for single-cell whole-genome sequencing, the collection of primers comprising a plurality of primer sets, wherein each primer set comprises one or more primers comprising a unique barcode region, wherein the unique barcode region comprises: i) A, T, and G nucleotides but not C nucleotides; ii) at least three base pairs separating any two neighboring A nucleotides; iii) at least a three base pair difference between the unique barcode region of one set of primers and the unique barcode region of any other set of primers in the collection; and/or iv) no sequential G nucleotides of five or more in a row.

In some embodiments, the unique barcode region is selected from an 8mer, 9mer, 10mer, 11mer, 12mer, 13mer, 14mer, 15mer, 16mer, 17mer, 18mer, 19mer and a 20mer. In some embodiments, the unique barcode region is an 11mer, a 12mer, a 13mer, a 14mer, a 15mer, a 16mer, a 17mer, an 18mer, a 19mer or a 20mer. In some embodiments, the unique barcode region is an 11mer.

In some embodiments, each primer set comprises at least two or more primers, wherein each of the two or more primers comprises the same common sequence, wherein the common sequence comprises the unique barcode region. In some embodiments, the common sequence is a 27mer. In some embodiments, the unique barcode region is located anywhere in the 27mer sequence from the fourth nucleotide from the 5′ end to the fourth nucleotide from the 3′ end. In some embodiments, the unique barcode is located in the 27mer sequence beginning from the fourteenth nucleotide from the 5′ end.

In some embodiments, each primer set comprises three primers, wherein each of the three primers comprises a common 27mer sequence comprising the unique bar code region; wherein one of the primers further comprises the sequence NNN-NNT-TT at the 3′ end of the common 27mer and wherein one of the primers further comprises the sequence-NNN-NNG-GG at the 3′ end of the common 27mer, wherein each N is randomly selected from A, T, G, and C.

In some embodiments, the presently disclosed subject matter provides a kit comprising two or more primer sets as described herein.

Accordingly, it is an object of the presently disclosed subject matter to provide methods for single cell whole genome sequencing, as well as to provide related primer collections and kits. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and Examples.

### BRIEF DESCRIPTIONS OF THE FIGURES

The presently disclosed subject matter can be better understood by referring to the following figures. The drawings are not intended to limit the scope of this presently disclosed subject matter, which is set forth with particularity in the claims as appended or as subsequently amended, but merely to clarify and exemplify the presently disclosed subject matter.

FIG. 1 is a schematic diagram showing a mechanism and workflow for multiple annealing and looping-based amplification cycles (MALBAC).

FIG. 2 is an image of a gel showing microsatellite marker polymerase chain reaction (PCR) results on MALBAC performed with 27mer or with a 17mer.

FIG. 3 is an image of agarose gel electrophoresis on modified MALBAC primers with 17mer during final amplification. Lanes 4 and 10 had primers with 5 G nucleotides in a series (not working). Lanes 14 and 15 show DNA distribution when using 17mer vs. 27mer during the final amplification stage.

FIG. 4 shows the design of combinatorially indexed DNA fragments in the final sequencing library.

### DETAILED DESCRIPTION

Genomic studies of DNA from single cells often rely on amplifying the DNA using polymerases, preparing DNA sequencing library for each cell, and performing DNA sequencing on the prepared library. Typically, preparation of a DNA sequencing library is done separately for each cell. The cost for library preparation increases rapidly with the number of cells. Although the cost of actual DNA sequencing has become cheaper every year, library preparation costs have remained approximately the same for the last decade.

The presently disclosed subject matter provides a new method for tagging DNA molecules from each individual single cell with a unique DNA barcode during DNA amplification with a polymerase. For instance, an exemplary set of 96 different barcodes can be used to tag 96 different single cells. Then, the barcoded amplicons from the 170 single cells can be pooled for to prepare a single DNA sequencing library, reducing the cost of library preparation by at least about 100-fold. Computational algorithms can be used to separate the sequenced DNA molecules from the different cells based on the barcode sequences, assigning them back to the original single cell. Based on the sizes of potential barcode sets, the presently disclosed methods provide accessible approaches to sequencing hundreds to thousands of single cells at a time.

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The terms “amplifying” or “amplification” as used herein refer to the process of creating nucleic acid strands that are identical or complementary to a complete target nucleic acid sequence, or a portion thereof. The term “identical” as used herein refers to a nucleic acid having the same or substantially the same nucleotide sequence as another nucleic acid.

As used herein, the term “linear amplification” denotes that the products of amplification are directly copied from original templates, so the increase of DNA products is linear. In contrast, for nonlinear amplifications, the products are copied from both original templates and the copied products. Polymerase chain reaction (PCR), for example, is a typical nonlinear amplification with exponential increase of products.

As used herein, the term “amplicon” refers to a polynucleotide that is a source and/or a product of an amplification process, e.g., polynucleotides that are products of a linear amplification process and which can be used as templates in an exponential amplification process (e.g., PCR). As used herein, the term “semi-amplicon” refers to a polynucleotide generated by extension with a primer sequence on only one end. It is a half product of a full-amplicon. As used herein, the term “full-amplicon” refers to polynucleotides with primer sequences (different or complementary with each other) on two ends, ready for PCR amplification.

The term “amplification reagents” refers to those reagents (deoxyribonucleotide triphosphates, buffer, etc.) used in an amplification reaction except for primers, nucleic acid template, and the amplification enzyme. Typically, amplification reagents along with other reaction components are placed and contained in a reaction vessel (test tube, microwell, etc.).

As used herein, the term “barcode” refers to a nucleic acid sequence that can be used to identify a sample or source of the nucleic acid material. Thus, where nucleic acid samples are derived from multiple sources (e.g., multiple single cells), the nucleic acids in each nucleic acid sample are in some instances tagged with different nucleic acid tags such that the source of the sample can be identified. Barcodes are also referred to as indexes or tags.

The term “cycle” when used in reference to a polymerase-mediated amplification reaction is used herein to describe steps of dissociation (“melting” or “de-annealing”) of at least a portion of a double stranded nucleic acid (e.g., a template from an amplicon, or a double stranded template, denaturation); hybridization of at least a portion of a primer to a template (annealing); and extension of the primer to generate an amplicon. In some instances, the temperature remains constant during a cycle of amplification (e.g., an isothermal reaction). In some instances, the number of cycles is directly correlated with the number of amplicons produced. In some instances, the number of cycles for an isothermal reaction is controlled by the amount of time the reaction is allowed to proceed.

When used in reference to nucleic acids, the terms “extend”, “extending”, “extension” and other variants, refer to incorporation of one or more nucleotides into a nucleic acid molecule. Nucleotide incorporation comprises polymerization of one or more nucleotides into the terminal 3′ OH end of a nucleic acid strand, resulting in extension of the nucleic acid strand. Nucleotide incorporation can be conducted with natural nucleotides and/or nucleotide analogs. Typically, but not necessarily, nucleotide incorporation occurs in a template-dependent fashion. Any suitable method of extending a nucleic acid molecule may be used, including primer extension catalyzed by a DNA polymerase or RNA polymerase.

The term “genome” as used herein is defined as the collective gene set carried by an individual, cell, or organelle. The term “genomic DNA” as used herein is defined as DNA material comprising the partial or full collective gene set carried by an individual, cell, or organelle.

The term “hybridization,” “hybridize,” “anneal” or “annealing” as used herein refer to the ability, under the appropriate conditions, for nucleic acids having substantial complementary sequences to bind to one another by Watson & Crick base pairing. Nucleic acid annealing or hybridization techniques are well known in the art. See, e.g., Sambrook, et al., Molecular Cloning: A Laboratory Manual, Second Edition, Cold Spring Harbor Press, Plainview, N.Y. (1989); Ausubel, F. M., et al., Current Protocols in Molecular Biology, John Wiley & Sons, Secaucus, N.J. (1994). The term “substantial complementary” as used herein refers both to complete complementarity of binding nucleic acids, in some cases referred to as an identical sequence, as well as complementarity sufficient to achieve the desired binding of nucleic acids. Correspondingly, the term “complementary hybrids” encompasses substantially complementary hybrids.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and chimeric forms containing DNA and RNA. Nucleic acids can be single-stranded or double-stranded.

The term “nucleotides” and related terms refer to a molecule comprising an aromatic base, a five-carbon sugar (e.g., ribose or deoxyribose), and at least one phosphate group. Canonical or non-canonical nucleotides are consistent with use of the term. The phosphate in some embodiments comprises a monophosphate, diphosphate, or triphosphate, or corresponding phosphate analog. In some embodiments, the nucleotide comprises 1, 2, 3, 4, 5, 6, 7, 8, 9 or 10 phosphate groups. The term “nucleoside” refers to a molecule comprising an aromatic base and a sugar.

The term “polymerase” and its variants, as used herein, refer to an enzyme that can catalyze polymerization of nucleotides (including analogs thereof) into a nucleic acid strand. Typically, such nucleotide polymerization can occur in a template-dependent fashion. Generally, a polymerase comprises one or more active sites at which nucleotide binding and/or catalysis of nucleotide polymerization can occur.

As used herein, the term “primer” refers to an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually, primers are extended by a polymerase. Primers usually have a length in the range of between 3 to 36 nucleotides, also 5 to 24 nucleotides, also from 14 to 36 nucleotides.

As used herein, the term “sequencing” and its variants comprise obtaining sequence information from a nucleic acid strand, typically by determining the identity of at least some nucleotides (including their nucleobase components) within the nucleic acid template molecule. While in some embodiments, “sequencing” a given region of a nucleic acid molecule includes identifying each and every nucleotide within the region that is sequenced, in some embodiments “sequencing” comprises methods whereby the identity of only some of the nucleotides in the region is determined, while the identity of some nucleotides remains undetermined or incorrectly determined. Any suitable method of sequencing may be used. In some embodiments, sequencing includes massively parallel sequencing platforms that employ sequence-by-synthesis, sequence-by-hybridization or sequence-by-binding procedures.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. The cells can be haploid cells or diploid cells. Furthermore, in general, cells from any population can be used in the methods, such as a population of prokaryotic or eukaryotic single celled organisms including bacteria or yeast. Methods described herein may require isolation of single cells for analysis. Any method of single cell isolation may be used, such as mouth pipetting, micro pipetting, flow cytometry/FACS, microfluidics, methods of sorting nuclei (tetraploid or other), or manual dilution. Such methods can be aided by additional reagents and steps, for example, antibody-based enrichment (e.g., circulating tumor cells), other small-molecule or protein-based enrichment methods, or fluorescent labeling. Single cells can be placed in any suitable reaction vessel in which single cells can be treated individually. For example, a 96-well plate, such that each single cell is placed in a single well. A single cell can also be placed in a PCR tube.

The term “template nucleic acid”, “template polynucleotide”, “target nucleic acid” “target polynucleotide”, “template strand” and other variations refer to a nucleic acid strand that serves as the basis nucleic acid molecule for generating a complementary nucleic acid strand. The template nucleic acid can be single-stranded or double-stranded, or the template nucleic acid can have single-stranded or double-stranded portions. The sequence of the template nucleic acid can be partially or wholly complementary to the sequence of the complementary strand. The template nucleic acid can be obtained from a naturally-occurring source, recombinant form, or chemically synthesized to include any type of nucleic acid analog.

### II. Methods and Compositions for Amplifying and Identifying Genomic DNA from Single Cells

In some aspects, provided herein is an indexed MALBAC (Multiple Annealing and Looping-Based Amplification Cycles) WGA method, which includes barcoding and pooling amplicons from dozens to hundreds of cells for one single sequencing library preparation. The pooled amplicons can be sequenced on either short- or long-read sequencing platforms. The obtained sequencing reads can be assigned to original cells based on a nucleotide index (i.e., a nucleotide “barcode”). Without the need for a special device, the disclosed methods provide for easy and cost-effective implementation of intermediate-throughput single cell whole-genome sequencing experiments. For example, the presently disclosed methods are cost-effective for implementation in the sequencing of individual single cells in samples comprising about 100 to about 1000 single cells.

The emergence of new cost-efficient next-generation sequencing (NGS) platforms has opened new doors for advancing research, especially in single-cell genomics. However, the availability of abundant genomic DNA content is critical for many available sequencing platforms; the same is valid for the majority of the downstream molecular biology assays. Often due to the nature of sampling or the availability of samples, the number of tissue/cells is limited. Several WGA methodologies have been developed to mitigate this shortcoming in the past few decades (Gawad et al., 2016; Hughes et al., 2005).

Single-cell genomics is one such study where the quantity of DNA is low. Single-cell genomics advances the understanding of genetics by increasing the resolution of genome study. With the improvement in genome sequencing, single-cell genome sequencing can be used in cancer research, haplotype analysis, and genomic variability. One particular technical challenge in the single-cell research field is to amplify a whole-genome by recovering a high percentage of the genome with a minimal amplification bias.

NGS includes sample generation, library preparation, sequencing, and bioinformatics. Even though next-generation sequencing has developed, reducing the costs per base, library preparation cost still takes a considerable portion of the overall expense of NGS. Studies centered around single-cell genomics are usually sample hungry, requiring hundreds of samples to make valid inferences. Especially when working with gametes, the studies need hundreds of gametes (sperms/eggs) to be sequenced.

Generating high-quality single cell sequencing data has four primary technical aspects: efficient physical isolation of individual cells; amplification of single cells to obtain sufficient material for downstream analyses; cost-effective analysis for hypothesis testing; and interpreting the data. The first step, isolating individual cells, is a rigorous study sector. With the advanced development in Fluorescent Activated Cell Sorting (FACS), single-cell isolation has become accessible to various sampling types.

The present disclosure focuses on the second step, where labs can efficiently amplify the low-content DNA to obtain enough starting material for further downstream analyses. As disclosed herein, the present disclosed subject matter also improves the third step by providing for a more cost-effective query of the genome by adding DNA barcodes to the single-cell DNA samples while performing WGA. Each unique barcode is associated with a single cell. This provides for the multiplexing of multiple single barcoded DNA to create a sequencing library that can be NGS sequenced as a single sample, thus drastically lowering the cost involved in the library preparation.

Multiplexing involves pooling and sequencing a large number of libraries simultaneously during a single run (Amini et al., 2014; Di et al., 2020; Rohland & Reich, 2012), reviewed in (Adey, 2021). The ultimate purpose is to increase sample throughput, reducing time and effort. While multiplexing, unique “barcode” or “tag” sequences are added to each DNA fragment during NGS library preparation. By way of example and not limitation, according to one embodiment, the presently disclosed subject matter also uses indices or barcodes where one unique barcode is added to a single sperm. In one example, ninety-six of these unique barcodes are used to tag a 96 well-plate consisting of one single sperm cell in each well. The tagging happens when a specially designed MALBAC primer containing a barcode is used to amplify a single sperm cell genome using the MALBAC procedure. Then standard library preparation and NGS can be performed. The revolutionary aspect of this procedure is that hundreds of individual sperm samples can be library prepped as a single sample, significantly reducing the cost (e.g., by 100-fold or more).

More particularly, according to conventional MALBAC methods (Zong et al. 2012) MALBAC comprises five cycles of linear amplification, making a “pre-amplification stage” and a final standard PCR amplification. See also, U.S. Patent Application Publication No. 2014/0200146, the disclosure of which is incorporated herein by reference in its entirety. The pre-amplification stage is initiated with a pair of quasi-degenerate primers, referred to as “NT” and “NG” primers, having a common sequence, e.g., a common 27mer sequence such as 5′-GTG AGT GAT GGT TGA GGT AGT GTG GAG-3′ (SEQ ID NO: 1), followed by a variable sequence that can evenly hybridize to DNA templates and create overlapped “semi-amplicons” throughout the whole genome. Five cycles of pre-amplification are performed, using a polymerase having strand displacing activity, e.g., Bst large fragment polymerase (New England Biolabs). A full amplicon is formed at the end of each cycle that loops at 58° C. due to the presence of complementary end sequences. This looping prevents further pre-amplification, reducing the overrepresentation of some genomic regions in the final pool of amplicons. After the pre-amplification stage, standard PCR amplification is performed with a single primer comprising the common sequence (e.g., the common 27mer) to generate 1-2 μg of DNA for use in downstream applications.

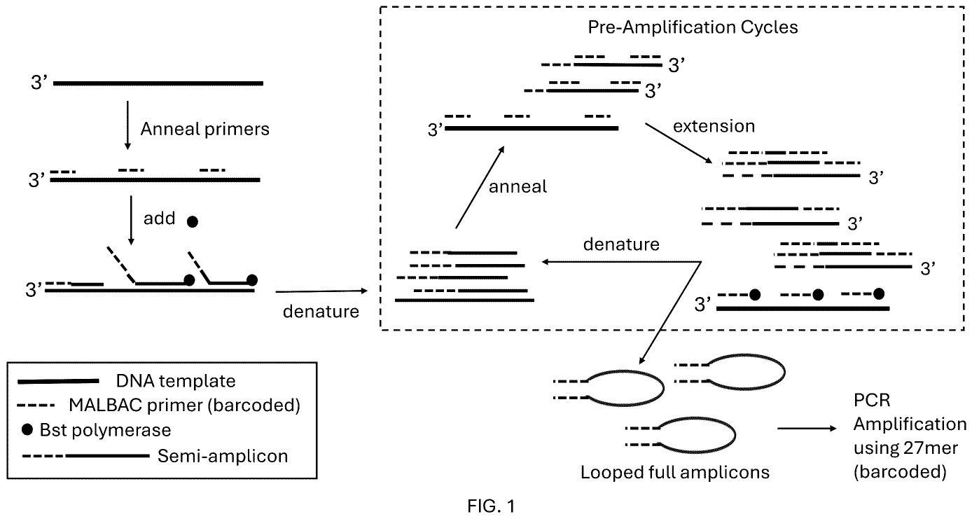

According to the presently disclosed subject matter, a unique barcode of 8 to 20 variable nucleotides is incorporated into the common sequence to provide barcoded MALBAC primers (also referred to herein as “indexed primers”). A pair of barcoded MALBAC primers (i.e., an indexed NT primer and an indexed NG primer) not only initiate the pre-amplification stage but also act to barcode the resulting amplicons. See FIG. 1. Then, standard PCR amplification is performed using the corresponding indexed common sequence, e.g., an “indexed 27mer”.

As described in the examples below, it was verified that deviation from the original common 27mer would amplify DNA as effectively as the original primers. For instance, an 8-bp region in the original 27mer common primer sequence was changed and MALBAC was performed using the primers shown in Table 1, below, using ˜1 picogram of genomic DNA as a template. The modified primers generated comparable DNA content (μg) compared to the original primers when observed in electrophoresis gel intensity and multiple microsatellite PCR. The indices (barcodes) were introduced in the first pre-amplification stage of MALBAC. The final stage (amplification with the corresponding indexed common 27mer) amplifies the generated full amplicons exponentially.

As further described in the examples below, a group of 8-bp (base pair) barcode indices (although as disclosed further herein, the barcodes can have varying lengths) was developed and these indices were incorporated in the 27mer to perform indexed MALBAC amplification (1-tier indexing). Each unique barcode is embedded in the 27mer portion of one set of indexed NT, NG, and 27mer primers and each primer set was used to label amplicons from a single cell. The uniquely labeled amplicons from 96 single sperm cells were pooled to provide one sequencing library preparation, where 2-tier indexing (via commercial standard library preparation) was introduced. Additional examples described hereinbelow show that the barcode can be present at different sites within the common sequence of the primers and the length of the barcode can be varied.

Thus, in some embodiments provided herein are single-cell whole-genome sequencing methods. The methods comprise amplifying nucleic acid (e.g., genomic DNA) from a single cell while simultaneously labeling the amplicons from each single cell with a unique barcode. For instance, in some embodiments, the method comprises: (a) amplifying nucleic acid from each of a plurality of single cells, wherein the amplification is performed using one or more first stage primers introduce a unique barcode region into amplicons from each of the plurality of single cells, wherein the unique barcode region introduced into the amplicons from each of the plurality of single cells differs from the unique barcode region introduced into amplicons from any other single cell in the plurality of single cells by at least three base pairs, thus providing amplicons from each of the plurality of single cells that are distinguishable based on the unique barcode assigned to each; and (b) further amplifying the amplicons from each single cell using a second stage primer comprising the same unique barcode region as the one or more first stage primers used during amplification of nucleic acid from the same cell, thereby providing further amplified amplicons from each of the plurality of cells. The further amplified amplicons can then be pooled and used to provide a single sequencing library preparation comprising amplicons from each of a plurality of cells. Demultiplexing the unique barcodes can identify the single cell used to produce the amplicons.

In some embodiments, the single sequencing library preparation is sequenced (e.g., via NGS) to provide a library of sequencing reads. The sequencing reads can be analyzed to identify one or more nucleic acids from one or more of the single cells based on the unique barcode sequence introduced during the amplification. For example, a cell of interest can be selected from the plurality of cells and the sequencing reads can be analyzed to identify reads containing the unique barcode introduced into amplicons of the cell of interest. In some embodiments, the sequencing reads can be analyzed to identify one or more nucleic acids from each of at least two of the plurality of single cells. In some embodiments, the reads can be analyzed to determine the sequence of at least 50%, of the genomic DNA from a single cell.

In some embodiments, the plurality of cells comprises at least 50 cells, or at least 100 cells. In some embodiments, the plurality of cells comprises about 100 cells to about 1000 cells. Typically, prior to step (a), a plurality of reaction vessels is provided wherein each reaction vessel comprises genomic DNA from a single cell. In some embodiments, each reaction vessel comprises single-stranded DNA provided by lysing a single cell to provide genomic DNA and then melting the genomic DNA. Step (a) is then performed by adding the one or more first stage primers comprising a unique barcode, a polymerase, and any other amplification reagents for the amplification reactions to each of the reaction vessels. The reaction vessels can be, for example, tubes or individual wells in a multi-well plate.

In some embodiments, the unique barcode region comprises A, T, and G nucleotides, but not C nucleotides. In some embodiments, the barcode region comprises at least three base pairs separating any two neighboring A nucleotides and/or is free of any sequential G nucleotide sequences of five or more in a row. In some embodiments, the unique barcode region introduced into the amplicons from each of the plurality of single cells differs from the unique barcode region introduced into amplicons from any other single cell in the plurality of single cells by at least four base pairs or by at least five base pairs. In some embodiments, the unique barcode region is between 8 and 20 nucleotides long. Thus, the unique barcode region can be an 8mer, a 9mer, a 10mer, an 11mer, a 12mer, a 13mer, a 14mer, a 15mer, a 16mer, a 17mer, an 18mer, a 19mer or a 20mer. In some embodiments, the unique barcode region is at least 10 nucleotides long. In some embodiments, the unique barcode region is a 10mer, an 11mer or a 12mer. In some embodiments, the unique barcode region is an 11mer.

The one or more first stage primers and the second stage primer used for each individual single cell can all comprise the same common sequence where the common sequence comprises the unique bar code region. Thus, the common sequence is a longer sequence than the barcode sequence and typically, the barcode region stops or starts at least two nucleotides or more, at least three nucleotides or more, or at least four nucleotides or more from the 5′ and/or the 3′ end of the common sequence. In some embodiments, the common sequence comprises 18 to 30 nucleotides. In some embodiments, the common sequence is an 18mer, a 19mer, a 20mer, a 21mer, a 22mer, a 23mer, a 24mer, a 25mer, a 26mer, a 27mer, a 28mer, a 29mer, or a 30mer. In some embodiments, the common sequence is a 27mer. In some embodiments, e.g., in a 27mer common sequence, the unique barcode region starts at least four nucleotides from the 5′ end of the common sequence and ends at least four nucleotides from the 3′ end of the common sequence. In some embodiments, the common sequence is a 27mer and the unique barcode region begins at the fourteenth nucleotide from the 5′ end of the common sequence.

In some embodiments, the common sequence is selected from: GTG-AGT-GAT-GGD-DDD-DDD-DGT-GTG-GAG (SEQ ID NO: 2); GTG-AGT-GAT-GGT-TGA-GGD-DDD-DDD-DAG (SEQ ID NO: 5); and GTG-AGT-GAT-GGT-TDD-DDD-DDD-DDD-GAG (SEQ ID NO: 10); wherein in each of SEQ ID NOs: 2, 5, and 10, D is A, T, and G nucleotides but not C; there are at least three base pairs separating any two neighboring A nucleotides; there are no sequential G nucleotides of five or more in a row; and there is an at least 4-bp difference between any two sequences of SEQ ID NO: 2, between any two sequences of SEQ ID NO: 5, or between any two sequences of SEQ ID NO: 10.

In some embodiments, the further amplifying of step (b) comprises PCR. PCR is well known in the art. PCR can be performed to exponentially amplify the amplicons from step (a). In some embodiments, step (b) comprises adding an excess of deoxyribonucleoside triphosphates to the amplicons from step (a) along with a DNA polymerase, e.g., a Taq polymerase. In some embodiments, step (a) comprises linear amplification, e.g., using a polymerase with strand displacement, e.g., Bst Polymerase ¢29 Polymerase, Vent Polymerase, Pyrophage 3173, Deep Vent Polymerase, TOPOTaq DNA polymerase, etc. In some embodiments, the method comprises MALBAC, e.g., (a) comprises a pre-amplification stage consisting of five cycles of linear amplification of an oligonucleotide of a single cell and (b) comprises a second amplification stage consisting of polymerase chain reaction (PCR) amplification.

In some embodiments, the presently disclosed subject matter provides an indexed MALBAC WGA method for single-cell whole-genome sequencing, the method comprising: providing a plurality of single cells for which whole-genome sequencing is desired; providing a plurality of primer sets, wherein each primer set comprises one or more primers, wherein the one or more primers of each primer set comprise a unique barcoded region that differs from the unique barcoded region of primers in any other one of the plurality of primer sets by 3 or more nucleotides, and wherein a single primer set is assigned to each of the plurality of single cells. The method further comprises performing MALBAC WGA to amplify one or more oligonucleotides of each of the plurality of single cells, using a single primer set for the MALBAC WGA of each of the plurality of cells to thereby introduce a unique barcode region into each amplicon of the amplified oligonucleotides of each of the single cells; pooling the barcoded amplicons from each of the single cells into a single sequencing library preparation; sequencing the library preparation to provide a plurality of sequencing reads; and analyzing the sequencing reads to identify genomic DNA sequences from single cells based on the unique barcode region. In some embodiments, the plurality of single cells comprises at least about 50 or more single cells. In some embodiments, the plurality of single cells comprises about 100 or more cells, 200 or more cells, 300 or more cells, 400 or more cells, 500 or more cells 750 or more cells, 1000 or more cells, 1500 or more cells, 2000 or more cells, or 5000 or more cells. In some embodiments the plurality of single cells comprises 100 to 1000 single cells.

In some embodiments, the presently disclosed subject matter provides a collection of primers for single-cell whole-genome sequencing, the collection of primers comprising a plurality of primer sets, wherein each primer set comprises one or more primers comprising a unique barcode region, wherein the unique barcode region comprises: i) A, T, and G nucleotides but not C nucleotides; ii) at least three base pairs separating any two neighboring A nucleotides; iii) at least a three base pair difference between the unique barcode region of one set of primers and the unique barcode region of any other set of primers in the collection; and/or iv) no sequential G nucleotides of five or more in a row.

The collection of primers can include at least about 50 primer sets, at least about 90 primer sets, at least about 100 primer sets, at least about 150 primer sets, at least about 200 primer sets, at least about 500 primer sets, or at least about 1000 primer sets. In some embodiments, the unique barcode region is a polynucleotide that is between about 8 and about 20 nucleotides in length. Thus, the unique barcode region can be selected from the group comprising an 8mer, a 9mer, a 10mer, an 11mer, a 12mer, a 13mer, a 14mer, a 15mer, a 16mer, a 17mer, an 18mer, a 19mer and a 20mer. In some embodiments, the unique barcode region is at least an 10mer or at least an 11mer (i.e., comprises 10 to 20 nucleotides or 11 to 20 nucleotides). In some embodiments, the unique barcode region is an 11mer.

In some embodiments, each primer set comprises at least two or more primers that comprise the same common sequence (and thus the same unique barcode region). In some embodiments, the common sequence is a 27mer. The unique barcode region is typically located anywhere in the 27mer sequence from the fourth nucleotide from the 5′ end of the common sequence to the fourth nucleotide from the 3′ end of the common sequence. Thus, in some embodiments, there are at least 3 nucleotides between the 5′ end of the barcode and the 5′ end of the common sequence and at least 3 nucleotides between the 3′ end of the barcode and the 3′ end of the common sequence. In some embodiments, the unique barcode region begins at eight, ninth, tenth, eleventh, twelfth, thirteenth or fourteenth nucleotide from the 5′ end of the common sequence.

In some embodiments, each primer set comprises three primers, e.g., two quasi-degenerate primers designed for use in a pre-amplification stage of MALBAC and a third primer for use in a second stage amplification (e.g., a PCR amplification). Thus, in some embodiments, each of the three primers comprise a common 27-mer sequence comprising the unique bar code region; one of the primers (i.e., an indexed NT primer) further comprises the sequence NNN-NNT-TT at the 3′ end of the common 27-mer, and one of the primers (i.e., an indexed NG primer) further comprises the sequence-NNN-NNG-GG at the 3′ end of the common 27-mer. In the NNN-NNT-TT and NNN-NNG-GG sequences, each N is randomly selected from A, T, G, and C.

In some embodiments, the presently disclosed subject matter provides a kit for use in performing a method as described herein. For example, the kit can comprise at least two or more (e.g., about 10 or more, about 50 or more, about 100 or more, about 200 or more, about 500 or more, or about 1000 or more) primer sets as described herein. The kit can include polymerases and additional reagents for use in the amplification of nucleic acids (e.g., buffers, salts, nucleotides, and so forth), for obtaining single cells, for lysing single cells, and/or for performing sequencing library preparation.

The kit components can be provided in a suitable container or containers, e.g., so that individual components can be placed in separate containers. In some embodiments, the kit can include one or more vessels for performing a step or steps of the presently disclosed methods.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for the Examples

### Whole-Genome Amplification.

To obtain enough DNA from each sperm for genotyping, lysed single sperm cells were used for MALBAC (multiple annealing and looping-based amplification) whole-genome amplification (Zong et al., 2012). MALBAC consists of a pre-amplification stage and a standard PCR amplification. The preamplification is initiated with a pair of primers, each having a common 27-nucleotide sequence (e.g., 5′-GTG AGT GAT GGT TGA GGT AGT GTG GAG-3′ (SEQ ID NO: 1)) as well as an eight-nucleotide variable sequence comprising five variable nucleotides that can evenly hybridize to the templates. The MALBAC protocol was unchanged other than the use of indexed 27mers, and their corresponding indexed NG and NT primer sequences, e.g., barcoded primers as described in Table 1, below. One set of primers (i.e., one unique indexed 27mer and its corresponding indexed NG and NT primers) was used for each individual cell.

Standard PCR amplification. The standard touchdown PCR was used to evaluate the presence of different microsatellite markers. The thermal cycling program for microsatellite amplification consisted of 3 min at 95° C., ten cycles of 35 s at 95° C., 35 s at 56° C. (the temperature increased by 1° C. for each cycle) and 45 s at 72° C., 30 cycles of 35 s at 95° C., 35 s at 48° C., 45 s at 72° C. and a final 10 min at 72° C.

### Example 1

Experiments were conducted to develop a Python computer program (MALBAC Barcodes R Us) selecting barcodes using the principles herein. Approximately 460 unique 8-bp barcodes are identified. Of note, initial testing began with 8-bp barcodes, but as disclosed herein, barcodes can range in size from 8 base pairs (bp) to 20 bp. Next, 12 different sets of indexed NT, NG, and 27mers were synthesized, with barcodes located between sites 12 and 19 in the 27mer sequence (see Table 1, below), and MALBAC was performed using these primers on ˜0.5 picogram of DNA (similar to the amount of DNA in a Daphnia sperm). “N” in the sequences of Table 1 can be any one of G, T, A, and C. “D” in Table 1 refers to A, T, or G, but not C wherein (a) there are at least three bps between any 2 neighboring As, (b) there is at least a 4 bp difference with any other barcode, and (c) where there are no sequential G sequences of five of more. The amplification with all sets of primers was successful, yielding ˜1 μg of DNA amplicon. The amplification coverage of these indexed primers across the genome is equivalent to the original primers. Regular PCR for 12 randomly selected loci using these MALBAC products as templates showed that these loci are all present in the MALBAC amplicons.

TABLE 1
Indexed MALBAC Primers with 8 bp barcode between locations 12 and 19
of the 27 mer.

| Primer | Sequence | SEQ ID NO: |
|---|---|---|
| Indexed | 5′-GTG-AGT-GAT-GGD-DDD-DDD-DGT-GTG-GAG-3′ | 2 |
| 27 mer-1 | | |
| Indexed | 5′-GTG-AGT-GAT-GGD-DDD-DDD-DGT-GTG-GAG-NNN- | 3 |
| NT-1 | NNT-TT3′ | |
| Indexed | 5′-GTG-AGT-GAT-GGD-DDD-DDD-DGT-GTG-GAG-NNN- | 4 |
| NG-1 | NNG-GG-3′ | |

A plurality of sets of 8-bp indexed 27mer, NT, and NG primers were synthesized for further testing. To be selected as effective barcodes, a set of barcoded primers had to generate ˜1 μg DNA amplicon and >90% genome-wide amplification coverage (PCR test of 24 loci, 2 on each chromosome). Testing was also conducted by placing the barcodes at other locations in the 27mer and/or changing the barcode length (e.g., 10-bp index, 11-bp index, etc.).

To implement the 2nd-level indexing, barcoded library preparation was performed on a pool of indexed MALBAC product of 384 single sperm from Daphnia genotype PA42, for which a single-sperm based genetic map is published for benchmarking (Xu et al., 2015.). The pooled DNA was end-repaired, size-selected, and ligated with a barcode index, e.g., an Illumina index as shown in FIG. 4. The DNA fragmentation step was skipped to avoid removing the 27mer barcodes/index from the DNA molecules. This is generally not a problem for the sequencing library as the normal fragment size of MALBAC products is between 300-1000 bp. Using 150-bp paired-end reads, each sperm was sequenced at 5× genome-wide coverage (the coverage used in previous single-sperm genetic map). An alternative strategy was also developed where barcoded PacBio direct ligation library preparation was used (directly ligating barcoded adapters to the amplicons) and sequencing was performed on a PacBio Sequel II platform.

After sequencing, raw reads were de-multiplexed into individual sperm (after de-multiplexing using barcodes) based on the MALBAC barcodes by using a de-multiplexing software. Several highly flexible packages are available, e.g., FreeBarcodes (Hawkins et al., 2018). After demultiplexing, a genetic map was built based on 96 randomly selected sperm using the disclosed bioinformatic procedure. Using the presently disclosed sequencing approach, the amount of sequenced genomic regions and genomic coverage was comparable to sequencing performed with individually sequence sperm cells.

### Example 2

### Generation of Indexed MALBAC Primers Preserving the Nature of MALBAC

Indexed MALBAC primers were created. The initial design parameters for such primers include: 1) strictly exclude C nucleotides in the barcode, and A, T, and G nucleotides must be all be present to maximize the randomness; 2) two neighboring A nucleotides in the barcode must be at least three bp apart so that the triple Ts of NT primer would not bind to the 27mer; 3) each barcode varies from the remaining barcodes by at least two nucleotides, which will allow accurate de-multiplexing in case if sequencing errors occur in the barcoded region; and 4) no five or more G nucleotides in a row are allowed in the primer, as the initial tests reflected that five or more stretches of G nucleotides inhibit the MALBAC.

A Python computer program (MALBAC Barcodes R′ Us) was developed to select the barcodes following these parameters, generating approximately 300 valid unique 8-bp barcodes. From there, 96 different sets of indexed NT, NG, and 27mers were synthesized, with barcodes located between sites 18 and 25 in the 27mer sequence. See Table 2, below. “N” in the sequences of Table 2 can be any one of G, T, A, and C. “D” in Table 2 refers to A, T, or G, but not C wherein (a) there are at least three bps between any 2 neighboring As, (b) there is at least a 4 bp difference with any other barcode, and (c) where there are no sequential G sequences of five of more.

TABLE 2
Indexed MALBAC Primers with 8 bp barcode between locations 18 and 25
of the 27 mer.

| Primer | Sequence | SEQ ID NO: |
|---|---|---|
| Indexed | 5′-GTG-AGT-GAT-GGT-TGA-GGD-DDD-DDD-DAG-3′ | 5 |
| 27 mer-2 | | |
| Indexed | 5′-GTG-AGT-GAT-GGT-TGA-GGD-DDD-DDD-DAG-NNN- | 6 |
| NT-2 | NNT-TT3′ | |
| Indexed | 5′-GTG-AGT-GAT-GGT-TGA-GGD-DDD-DDD-DAG-NNN- | 7 |
| NG-2 | NNG-GG-3′ | |

An example of a particular indexed 27mer-2 of Table 2 is 5′-gtg-agt-gat-ggt-tga-ggT-TGT-AGT-Tag-3′ (SEQ ID NO: 8), where the barcode is shown in uppercase letters.

### Example 3

### Testing the Synthesized MALBAC Primers

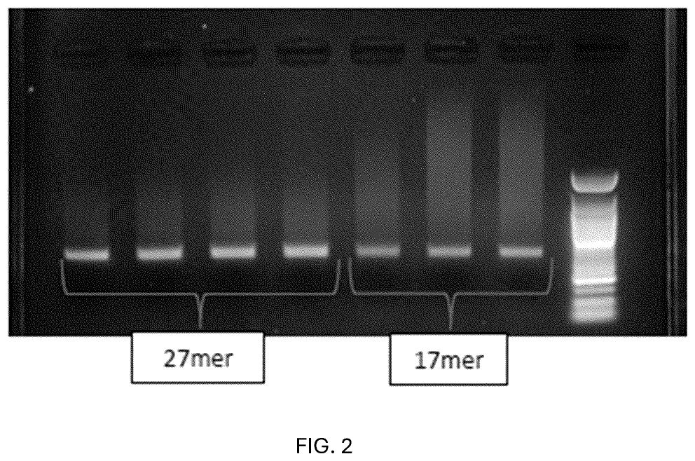

MALBAC was performed using the synthesized primers on ˜1 picogram of DNA (similar to the amount of DNA in a Daphnia sperm). The amplification with all sets of primers was successful, yielding ˜1 μg of DNA amplicon. The amplification coverage of these indexed primers across the genome is equivalent to the original primers. Regular PCR for 12 randomly selected loci from each chromosome using these MALBAC products as templates showed that these loci are all present in the MALBAC amplicons. See FIG. 2.

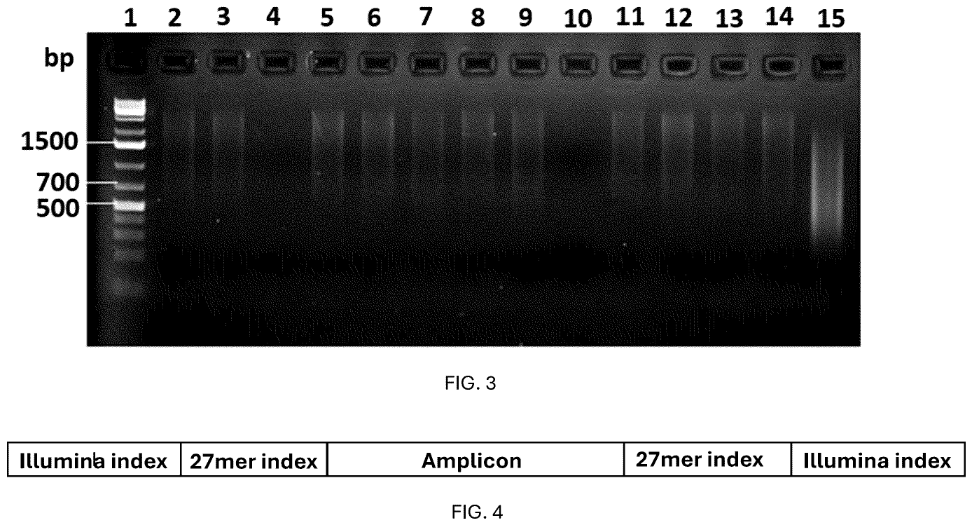

To further ease the MALBAC process, a study was performed to test if a common stretch of 27mer present in all the modified NG and NTs can exponentially amplify the full amplicons. An oligomer containing only the common 5′ stretch GTG AGT GAT GGT TGA GG (SEQ ID NO: 9) that is 17 nucleotides long, referred to as “17mer” henceforth, was prepared. The strategy was to ease the laborious task of adding individual 27mer in all the 96 samples in a plate by adding a common 17mer that can be premixed in the PCR buffer. Using 17mer, MALBAC amplified the samples. However, compared to the original common 27mer used in the amplification stage, the 17mer generated almost half DNA content than did 27mer, and the size distribution was also different (bigger size generated by the 17mer than 27mer). See FIG. 3. The lesser DNA quantity generated by the 17mer would have been a minor problem because the DNA content was still enough for library preparation. Nevertheless, the bigger DNA size and distribution would mean that the DNA needed to be sheared for library preparation. In contrast, the DNA size distribution for the generated DNA from 27mer is around 600 bp, desirable for library preparation. See FIG. 3. However, a problem is that shearing can destroy the purpose of introducing the barcode during the MALBAC amplification. So, the strategy of using a common 17mer during the amplification stage was set aside.

### Example 4

### Discussion of Examples 1-3

With improvements in technology, DNA sequencing has reduced the cost to the point where the reagent cost of sample preparation is the limiting factor. The quality and quantity of sequence data required per sample is often less than the commercial cost of library preparation. The available library preparation kits have limited throughput, drastically increased cost when scaling to hundreds or thousands of samples. However, several published studies have presented a way to drastically reduce the cost associated with library preparation (Rohland & Reich, 2012). One strategy is to pool samples before library preparation to save funds and time, but the samples need to be indexed (barcoded) first.

The presently disclosed methods were designed to help examine the evolutionary forces in the evolution of recombination rate. Different theories converge on predicting that the transition to a novel environment will lead to an increased recombination rate due to novel selection pressure (Butlin, 2005). However, domesticated animals under strong directional selection seem to have no recombination rate increase (Munoz-Fuentes et al., 2015). Due to the lack of population-level recombination rate data, this hypothesis remains untested mainly in natural systems. In species with well-understood ecology, like Daphnia, the lack of population and individual level recombination rate data is a significant challenge for understanding how ecological shift and selection affect recombination rate evolution. Despite efforts to assess fine-scale recombination rate data on two different chromosomal segments (Neupane & Xu, 2020) being attainable, it is still laborious to scale up.

Analysis to sequence 96 sperms for 50 individuals can involve making ˜5,000 libraries. Some library low-cost construction methods like RIPTIDE (genomx.com/product/riptide. Accessed November 2021) still costs about $10 per sample, resulting in a cost for library construction of almost $50,000. Development of a novel 2-tier combinatorial indexing single-cell sequencing approach can reduce library costs by a hundred-fold. The presently disclosed subject matter focuses on the first-tier indexing, which is accomplished by barcoding the DNA amplicon of each cell using re-engineered MALBAC amplification. The 2-tier indexing occurs through barcoded sequencing library construction using commercially available kits. MALBAC costs ˜$0.7 per sample and provides an even amplification coverage across the genome (Zong et al., 2012), hence, making it an attractive choice.

Herein it is described that modifying eight nucleotides in the common MALBAC primer 27mer can introduce a unique barcode indexed during MALBAC amplification itself. This poses a very efficient strategy to introduce barcodes in hundreds of single sperm samples, integrating it with a mandatory WGA step. First, when combined with indexing (introducing the first barcode during WGA), 96 single sperm cells can be grouped, pooled, and sequenced in a few runs. The first step of the 2-tier indexing was tested on ˜1 μg of genomic DNA. A potential concern is the differences in ligation or primer efficiency of different barcodes. A screening of 96 unique barcoded primers was performed that gave insight into whether primer efficiency varies. See FIG. 3. It is noteworthy to mention that one of the parameters of barcode design according to the presently disclosed subject matter, i.e., that having five or more G nucleotides in a series would reduce the primer efficiency was added to the primer design rules based on this study.

The presently disclosed subject matter can significantly increase throughput by streamlining the indexing and library preparation in 96-well plates, reducing the technician's time. The significant reduction of cost in multiplexing hundreds of samples to perform NGS would make it revolutionary because this method can readily be adopted across various model organisms, owing to the sensitivity and versatility of MALBAC. Lesser financial investment in sequencing hundreds of sperm samples breaks the barrier to effectively producing genetic maps that would otherwise be financially impractical. The whole streamlined workflow to implement 2nd-level indexing can be performed as described in Example 1. For example, standard barcoded Illumina library preparation eliminating the DNA fragmentation step on a pool of indexed MALBAC products of 96 single sperm cells from Daphnia species can be performed. After sequencing, the Illumina barcodes and then the MALBAC barcodes are de-multiplexed into individual sperm using open-source de-multiplexing software.

Goals of the presently disclosed subject matter include increased throughput and decreased reagent costs while building appropriate libraries for pooled sperm sequencing to generate genetic maps of multiple Daphnia species. However, the present methods are also applicable and adoptable by academic laboratories to create hundreds of barcoded libraries at a hundred magnitude less cost than the commercial cost of library preparation and to perform whole genome sequencing of other cell types.

### Example 5

Primers with 11-bp Barcodes

Additional studies were performed by changing the 8-bp barcode to an 11-bp barcode. Primer sets were designed with an 11-bp barcode between locations 14 and 24 of the canonical MALBAC 27mer See Table 3. “N” in the sequences of Table 3 can be any one of G, T, A, and C. “D” in Table 3 refers to A, T, or G, but not C wherein (a) there are at least three bps between any 2 neighboring As, (b) there is at least a 4 bp difference with any other barcode, and (c) where there are no sequential G sequences of five of more.

TABLE 3
Indexed MALBAC Primers with 11 bp Barcodes

| Primer | Sequence | SEQ ID NO: |
|---|---|---|
| Indexed | 5′-GTG-AGT-GAT-GGT-TDD-DDD-DDD-DDD-GAG-3′ | 10 |
| 27 mer-3 | | |
| Indexed | 5′-GTG-AGT-GAT-GGT-TDD-DDD-DDD-DDD-GAG-NNN- | 11 |
| NT-3 | NNT-TT-3′ | |
| Indexed | 5′-GTG-AGT-GAT-GGT-TDD-DDD-DDD-DDD-GAG-NNN- | 12 |
| NG-3 | NNG-GG-3′ | |

The indexed primers were used in the amplification of 104 individual cells. A library of the amplicons prepared using the primers was prepared and sequenced. The sequencing reads were analyzed. As it was observed that the first several bases of the MALBAC primer would often be left off the beginning or end of a read, an initial query of the reads was performed by searching for patterns that contained 11 bases sandwiched by “GGTT” (nucleotides at positions 10-13 of SEQ ID NO. 10) and “GAG” (nucleotides at positions 25-27 of SEQ ID NO: 10). The majority of reads (89.71%) had one matching barcode set. About 10% of reads had no matching barcode set, while <0.5% had more than one matching set. See Table 4, below.

TABLE 4
Reads with Matching Barcodes using “Trimmed” Sequence Query.

| | | Percent of |
|---|---|---|
| | # of Reads | Reads (%) |
| Reads with 0 matching barcode sets | 356,317 | 9.96 |
| Reads with 1 matching barcode sets | 3,209,548 | 89.71 |
| Reads with 2 matching barcode sets | 9,491 | 0.27 |
| Reads with 3 matching barcode sets | 1,951 | 0.05 |
| Reads with 4 matching barcode sets | 411 | 0.01 |
| Reads with 5 matching barcode sets | 68 | 0.00 |
| Reads with 6 matching barcode sets | 7 | 0.00 |
| Reads with 7 matching barcode sets | 2 | 0.00 |
| Reads with 8 matching barcode sets | 1 | 0.00 |

To test whether the number of matching barcodes is due to coincidental matches, the analysis was rerun only using the full barcoded primer sequences. This decreased the number of reads with >1 matching barcode set. See Table 5. This suggested that the barcode query can be stricter while still allowing for the potential of the MALBAC adapters being truncated at either end of a read.

TABLE 5
Reads with Matching Barcodes - Full Barcoded Sequences Query.

| | | Percent of |
|---|---|---|
| | # of Reads | Reads (%) |
| Reads with 0 matching barcode sets | 356,429 | 9.96 |
| Reads with 1 matching barcode sets | 3,219,502 | 89.99 |
| Reads with 2 matching barcode sets | 1,852 | 0.05 |
| Reads with 3 matching barcode sets | 13 | 0.00 |

To reduce the false positive rate while still accounting for truncated MALBAC adapters at the end of reads, the query was rerun as two separated searches: (1) a query conducted in regions 100 bp from the ends of the reads for an 11 bp sequence sandwiched by “GGTT” and “GAG” and (2) a query in the inner regions of the reads for an 11 bp sequence sandwiched by the full 13mer of positions 1-13 of the SEQ ID NO: 10 and “GAG” (i.e., of positions 25-27 of SEQ ID NO: 10). This resulted in a reduction of reads identified as having more than 1 matching barcode set. See Table 6, below. Many of the reads previously categorized as having more than 1 matching barcode set changed to having 0 matching barcodes sets as a result of the increased specificity of the modified query.

TABLE 6
Reads with Matching Barcodes - Modified Query

| | | Percent of |
|---|---|---|
| | # of Reads | Reads (%) |
| Reads with 0 matching barcode sets | 358,580 | 10.02 |
| Reads with 1 matching barcode sets | 3,210,556 | 89.74 |
| Reads with 2 matching barcode sets | 7,535 | 0.27 |
| Reads with 3 matching barcode sets | 987 | 0.05 |
| Reads with 4 matching barcode sets | 118 | 0.01 |
| Reads with 5 matching barcode sets | 18 | 0.00 |
| Reads with 6 matching barcode sets | 1 | 0.00 |
| Reads with 7 matching barcode sets | 1 | 0.00 |

The same pattern was observed when the analysis was rerun to only include the canonical sequences. This provided 358,599 reads (10.02%) with 0 matching barcode sets; 3,217,896 reads (89.94%) with 1 matching barcode set; 1,291 reads (0.04%) with 2 matching barcode sets; and 10 reads (0.00%) with 3 matching barcode sets.

Examination of the ˜10% of reads with 0 matching barcode sets suggested that the majority of these reads (˜70%, or ˜7% of the total reads) have a canonical barcode in one direction but lack any barcode going in the opposite direction. Without being bound to any one theory, this is believed to be due to degradation at one end of the amplicon. Reads without any barcodes were very low, accounting for 0.19% of all reads. Overall, the approximately 90% single matching barcode set results showcased the effectiveness of the indexing strategy.

### Example 6

### Protocols for Different Sequencing Platforms

The pooled amplicons produced by the presently disclosed methods were compatible with a variety of commercially available high-throughput sequencing platforms. For example, prior to sequencing using a PacBio platform, library preparation was performed using standard PacBio protocols. For use of the Illumina sequencing platform, a change was made from the standard library preparation protocol. More particularly, for Illumina library prep, 500 ng of DNA was used as input into the NEBNext Ultra II FS DNA Library Prep Kit (E7805). FS Enzyme Mix, which includes fragmentase, was added following the protocol. However, the 37° C. incubation step for optimal fragmentase activation was skipped. Instead, the mixture was immediately incubated at 67° C. for 30 minutes instead. The rest of the protocol is the same as the manufacturer's protocol.

While the present disclosure refers to certain embodiments, numerous modifications, alterations, and changes to the described embodiments are possible without departing from the sphere and scope of the present disclosure, as defined in the appended claim(s). Accordingly, it is intended that the present disclosure not be limited to the described embodiments, but that it has the full scope defined by the language of the following claims, and equivalents thereof. The discussion of any embodiment is meant only to be explanatory and is not intended to suggest that the scope of the disclosure, including the claims, is limited to these embodiments. In other words, while illustrative embodiments of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. Moreover, the following claims are hereby incorporated into this description by this reference, with each claim standing on its own as a separate embodiment of the present disclosure.

### REFERENCES

Adey, A. C. (2021). Tagmentation-based single-cell genomics. 1693-1705. doi.org/10.1101/gr.275223.121.31.
Amini, S., Pushkarev, D., Christiansen, L., Kostem, E., Royce, T., Turk, C., Pignatelli, N., Adey, A., Kitzman, J. O., Vijayan, K., Ronaghi, M., Shendure, J., Gunderson, K. L., & Steemers, F. J. (2014). Haplotype-resolved whole-genome sequencing by contiguity-preserving transposition and combinatorial indexing. Nature Genetics, 46 (12), 1343-1349. doi.org/10.1038/ng.3119.
Bagnell, C. R. (2005). Laser capture microdissection. Molecular Diagnostics: For the Clinical Laboratorian, 274 (November), 219-224. doi.org/10.1385/1-59259-928-1:219.
Blainey, P. C. (2013). The future is now: Single-cell genomics of bacteria and archaea. FEMS Microbiology Reviews, 37 (3), 407-427. doi.org/10.1111/1574-6976.12015.
Butlin, R. K. (2005). Recombination and speciation. Molecular Ecology, 14 (9), 2621-2635. doi.org/10.1111/j.1365-294X.2005.02617.x.
Di, L., Fu, Y., Sun, Y., Li, J., Liu, L., Yao, J., Wang, G., Wu, Y., Lao, K., Lee, R. W., Zheng, G., Xu, J., Oh, J., Wang, D., Sunney Xie, X., Huang, Y., & Wang, J. (2020). RNA sequencing by direct tagmentation of RNA/DNA hybrids. Proceedings of the National Academy of Sciences of the United States of America, 117 (6), 2886-2893. doi.org/10.1073/pnas. 1919800117.
Gawad, C., Koh, W., & Quake, S. R. (2016). Single-cell genome sequencing: Current state of the science. Nature Reviews Genetics, 17 (3), 175-188. doi.org/10.1038/nrg.2015.16.
Hawkins, J. A., Jones Jr., S. K., Finkelstein, I. J., Press, W. H. (2018). Indel-correcting DAN barcodes for high-throughput sequencing. Proc Natl Acad Sci USA, 115, E6217-E6226.
Hughes, S., Arneson, N., Done, S., & Squire, J. (2005). The use of whole genome amplification in the study of human disease. Progress in Biophysics and Molecular Biology, 88 (1), 173-189. doi.org/10.1016/j.pbiomolbio.2004.01.007.
Lan, F., Demaree, B., Ahmed, N., Abate, A. R. (2017). Single-cell genome sequencing at ultra-high-throughput with microfluidic droplet barcoding. Nat. Biotechnol. 35, 640-646. doi.org/10.1038/nbt.3880.
Lasken, R. S. (2013). Single-cell sequencing in its prime. Nature Materials, 12 (4), 367-376. doi.org/10.1038/nmat3550.
Munoz-Fuentes, V., Marcet-Ortega, M., Alkorta-Aranburu, G., Forsberg, C. L., Morrell, J. M., Manzano-Piedras, E., Soderberg, A., Daniel, K., Villalba, A., Toth, A., Di Rienzo, A., Roig, I., & Vila, C. (2015). Strong artificial selection in domestic mammals did not result in an increased recombination rate. Molecular Biology and Evolution, 32 (2), 510-523. doi.org/10.1093/molbev/msu322.
Navin, N. E. (2014). Cancer genomics: one cell at a time. Genome Biology, 15 (8), 452. doi.org/10.1186/s13059-014-0452-9.
Neupane, S., & Xu, S. (2020). Adaptive Divergence of Meiotic Recombination Rate in Ecological Speciation. Genome Biology and Evolution, 12 (10), 1869-1881. doi.org/10.1093/gbe/evaa182.
Rohland, N., & Reich, D. (2012). Cost-effective, high-throughput DNA sequencing libraries for multiplexed target capture. Genome Research, 22 (5), 939-946. doi.org/10.1101/gr.128124.111.
Shapiro, E., Biezuner, T., & Linnarsson, S. (2013). Single-cell sequencing-based technologies will revolutionize whole-organism science. Nature Reviews Genetics, 14 (9), 618-630. doi.org/10.1038/nrg3542.
Wang, Y., Waters, J., Leung, M. L., Unruh, A., Roh, W., Shi, X., Chen, K., Scheet, P., Vattathil, S., Liang, H., Multani, A., Zhang, H., Zhao, R., Michor, F., Meric-Bernstam, F., & Navin, N. E. (2014). Clonal evolution in breast cancer revealed by single nucleus genome sequencing. Nature, 512 (7513), 155-160. doi.org/10.1038/nature13600.
Xu, S., Ackerman, M. S., Long, H., Bright, L., Spitze, K., Ramsdell, J. S., Thomas, W. K., & Lynch, M. (2015). A male-specific genetic map of the microcrustacean Daphnia pulex based on single-sperm whole-genome sequencing. Genetics, 201 (1), 31-38. doi.org/10.1534/genetics. 115.179028.
Yin, Y., Jiang, Y., Lam, K. G., Berletch, J. B., Disteche, C. M., Noble, W. S., Steemers, F. J., Camerini-Otero, R. D., Adey, A. C., Shendure, J. (2019). High-Throughput Single-Cell Sequencing with Linear Amplification. Mol. Cell 76, 676-690 e610. doi.org/10.1016/j.molcel.2019.08.002.
Zong, C., Lu, S., Chapman, A. R., & Xie, X. (2012). Genome-wide detection of single-nucleotide and copy-number variations of a single human cell. Science, 338 (6114), 1622-1626. doi.org/10.1126/science.1227764.

What is claimed is:

1. A single-cell whole-genome sequencing method, the method comprising:
(a) amplifying nucleic acid from each of a plurality of single cells, wherein the amplification is performed using one or more first stage primers that introduce a unique barcode region into amplicons from each of the plurality of single cells, wherein the unique barcode region introduced into the amplicons from each of the plurality of single cells differs from the unique barcode region introduced into amplicons from any other single cell in the plurality of single cells by at least three base pairs, thus providing amplicons from each of the plurality of single cells that are distinguishable based on the unique barcode assigned to each;
(b) further amplifying the amplicons from each single cell using a second stage primer comprising the same unique barcode region as the one or more first stage primers used during amplification of nucleic acid from the same cell, thereby providing further amplified amplicons from each of the plurality of cells;
(c) pooling the further amplified amplicons from each of the plurality of cells into a single sequencing library preparation;
(d) sequencing the single sequencing library preparation to provide a library of sequencing reads; and
(e) analyzing the sequencing reads to identify one or more nucleic acid sequences from one or more of the plurality of single cells based on the unique barcode sequence.

2. The single-cell, whole-genome sequencing method of claim 1, wherein the barcode region comprises:
i) A, T, and G nucleotides but not C nucleotides;
ii) at least three base pairs separating any two neighboring A nucleotides; and
iii) no sequential G nucleotides of five or more in a row.

3. The single-cell whole-genome sequencing method of claim 2, wherein the unique barcode region introduced into the amplicons from each of the plurality of single cells differs from the unique barcode region introduced into amplicons from any other single cell in the plurality of single cells by at least four base pairs or by at least five base pairs.

4. The single-cell whole-genome sequencing method of claim 1, wherein the unique barcode region is selected from the group consisting of an 8mer, a 9mer, a 10mer, an 11mer, a 12mer, a 13mer, a 14mer, a 15mer, a 16mer, a 17mer, an 18mer, a 19mer and a 20mer.

5. The single-cell whole-genome sequencing method of claim 4, wherein the unique barcode region is an 11mer.

6. The single-cell whole-genome sequencing method of claim 1, wherein, for each individual single cell, the one or more first stage primers used in (a) and the second stage primer used in (b) each comprise the same common sequence, wherein the common sequence is a 27mer that comprises the unique barcode region.

7. The single-cell whole-genome sequencing method of claim 6, wherein the unique barcode is located anywhere in the 27mer common sequence from the fourth nucleotide from the 5′ end to the fourth nucleotide from the 3′ end.

8. The single-cell whole-genome sequencing method of claim 6, wherein the unique barcode is located in the 27-mer common sequence beginning at the fourteenth nucleotide from the 5′ end.

9. The single-cell whole-genome sequencing method of claim 1, wherein the second amplification stage comprises polymerase chain reaction (PCR).

10. The single-cell whole-genome sequencing method of claim 1, wherein the method comprises Multiple Annealing and Looping-Based Amplification Cycles (MALBAC), wherein (a) comprises a pre-amplification stage consisting of five cycles of linear amplification of an oligonucleotide of a single cell and (b) comprises a second amplification stage consisting of polymerase chain reaction (PCR) amplification.

11. An indexed Multiple Annealing and Looping-Based Amplification Cycles (MALBAC) whole genome amplification (WGA) method for single-cell whole-genome sequencing, the method comprising:
providing a plurality of single cells for which whole-genome sequencing is desired;
providing a plurality of primer sets, wherein each primer set comprises one or more primers, wherein the one or more primers of each primer set comprise a unique barcoded region that differs from the unique barcoded region of primers in any other one of the plurality of primer sets by 3 or more nucleotides, and wherein a single primer set is assigned to each of the plurality of single cells;
performing MALBAC WGA to amplify one or more oligonucleotides of each of the plurality of single cells, using a single primer set for the MALBAC WGA of each of the plurality of cells to thereby introduce a unique barcode region into each amplicon of the amplified oligonucleotides of each of the single cells; and
pooling the barcoded amplicons from each of the single cells into a single sequencing library preparation, sequencing the library preparation to provide a plurality of sequencing reads, and analyzing the sequencing reads to identify genomic DNA sequences from single cells based on the unique barcode region.

12. The method of claim 11, wherein the plurality of single cells comprises at least about 50 or more single cells.

13. The method of claim 12, wherein the plurality of single cells comprises about 100 to about 1000 single cells.

14. A collection of primers for single-cell whole-genome sequencing, the collection of primers comprising a plurality of primer sets, wherein each primer set comprises one or more primers comprising a unique barcode region, wherein the unique barcode region comprises:
i) A, T, and G nucleotides but not C nucleotides;
ii) at least three base pairs separating any two neighboring A nucleotides;
iii) at least a three base pair difference between the unique barcode region of one set of primers and the unique barcode region of any other set of primers in the collection; and/or
iv) no sequential G nucleotides of five or more in a row.

15. The collection of primers of claim 14, wherein the unique barcode region is selected from an 8mer, 9mer, 10mer, 11mer, 12mer, 13mer, 14mer, 15mer, 16mer, 17mer, 18mer, 19mer and a 20mer.

16. The collection of primers of claim 15, wherein the unique barcode region is an 11mer, a 12mer, a 13mer, a 14mer, a 15mer, a 16mer, a 17mer, an 18mer, a 19mer or a 20mer.

17. The collection of primers of claim 16, wherein the unique barcode region is an 11mer.

18. The collection of primers of claim 14, wherein each primer set comprises at least two or more primers, wherein each of the two or more primers comprises the same common sequence, wherein the common sequence comprises the unique barcode region.

19. The collection of primers of claim 18, wherein the common sequence is a 27mer.

20. The collection of primers of claim 19, wherein the unique barcode region is located anywhere in the 27mer sequence from the fourth nucleotide from the 5′ end to the fourth nucleotide from the 3′ end.

21. The collection of primers of claim 20, wherein the unique barcode is located in the 27mer sequence beginning from the fourteenth nucleotide from the 5′ end.

22. The collection of primers of claim 14, wherein each primer set comprises three primers, wherein each of the three primers comprises a common 27mer sequence comprising the unique bar code region; wherein one of the primers further comprises the sequence NNN-NNT-TT at the 3′ end of the common 27mer and wherein one of the primers further comprises the sequence-NNN-NNG-GG at the 3′ end of the common 27mer, wherein each N is randomly selected from A, T, G, and C.

23. A kit comprising two or more primer sets of claim 14.
